(19) United States
(12) Patent Application Publication
(10) Pub. No.: US 2008/0090848 A1
(43) Pub. Date: Apr. 17, 2008

(54) Substituted Purinyl Derivatives With Immunomodulator And Chemoprotective Activity And Use Alone Or With Medium-Chain Length Fatty Acids Or Glycerides

(76) Inventors: Christopher Penney, Pierrefonds (CA); Boulos Zacharie, Laval (CA); Jean Barabe, Montreal (CA); Pierre Laurin, Montreal (CA); Lyne Gagnon, Laval (CA)

Correspondence Address: NIXON & VANDERHYE, PC, 901 NORTH GLEBE ROAD, 11TH FLOOR, ARLINGTON, VA, 22203, US

(21) Appl. No.: 11/661,108

(22) PCT Filed: Sep. 2, 2005
(86) PCT No.: PCT/CA05/01343

§ 371 Date: Feb. 26, 2007

Related U.S. Application Data
(60) Provisional application No. 60/606,915, filed on Sep. 3, 2004.

(51) Int. Cl. A61K 31/52; A61P 35/00; A61P 39/00

(52) U.S. Cl. 514/263.4

(57) ABSTRACT

The present invention describes new biological activities of immunomodulating 6-substituted purinyl compounds which make them particularly useful during the treatment of cancer. Collectively, these new biological activities make these purinyl compounds useful chemoprotective agents for the treatment of myelosuppression which is associated with cancer chemotherapy and/or radiotherapy. This chemoprotective activity is in addition to the immunomodulating and subsequent anticancer activity displayed by these compounds. The chemoprotective usefulness of these compounds is further enhanced by the use of medium-chain fatty acids or salts or triglycerides or mono- or diglycerides in combination with the 6-substituted purinyl compounds of this invention.

### CROSS-REFERENCE TO RELATED APPLICATIONS

This application claims the benefit of provisional U.S. Appln. No. 60/606,915, filed Sep. 3, 2004; the contents of which are incorporated by reference herein.

### FIELD OF THE INVENTION

The present invention describes new biological activities of immunomodulating 6-substituted purinyl compounds which make them particularly useful during the treatment of cancer. Collectively, these new biological activities make these purinyl compounds useful chemoprotective agents for the treatment of myelosuppression which is associated with cancer chemotherapy and/or radiotherapy. This chemoprotective activity is in addition to the immunomodulating and subsequent anticancer activity displayed by these compounds. The chemoprotective usefulness of these compounds is further enhanced by the use of medium-chain fatty acids or salts or triglycerides or mono- or diglycerides in combination with the 6-substituted purinyl compounds of this invention.

### BACKGROUND OF THE INVENTION

Chemotherapy refers to the use of cytotoxic drugs such as, but not limited to, cyclophosphamide, doxorubicin, daunorubicin, vinblastine, vincristine, bleomycin, etoposide, topotecan, irinotecan, taxotere, taxol, 5-fluorouracil, methotrexate, gemcitabine, cisplatin, carboplatin or chlorambucil in order to eradicate cancer cells and tumors. However, these agents are non-specific and, particularly at high doses, they are toxic to normal, rapidly dividing cells. Ionizing radiation is also toxic to normal, rapidly dividing cells. This often leads to various side effects in patients undergoing chemotherapy and/or radiotherapy. Myelosuppression, a severe reduction of blood cell production in bone marrow, is one such side effect. It is characterized by anemia, leukopenia, neutropenia, agranulocytosis and thrombocytopenia. Severe chronic neutropenia is also characterized by a selective decrease in the number of circulating neutrophils and an enhanced susceptibility to bacterial infections.

The essence of treating cancer with chemotherapeutic drugs is to combine a mechanism of cytotoxicity with a mechanism of selectivity for highly proliferating tumor cells over host cells. However, it is rare for chemotherapeutic drugs to have such selectivity. The cytotoxicity of chemotherapeutic agents limits administrable doses, affects treatment cycles and seriously jeopardizes the quality of life for the cancer patient. Similar drawbacks affect the treatment of cancer with radiotherapy.

Anemia is a symptom of various diseases and disorders. It refers to that condition which exists when there is a reduction below normal in the number of red blood cells or erythrocytes, the quantity of hemoglobin, or the volume of packed red blood cells in the blood as characterized by a determination of the hematocrit. Hemoglobin is a tetrapeptide which binds and transports oxygen in the blood. Within the context of the current invention, it is of particular interest to address anemia associated with the use of chemotherapy or radiotherapy in the treatment of cancer. According to a statement published in BioWorld Today (page 4; Jul. 23, 2002), approximately 67% of cancer patients undergoing chemotherapy in the United States become anemic.

Cancer treatments often also result in a decrease of white blood cells or leukocytes. The resulting condition is referred to a leukopenia. More specifically, cancer treatments can result in a decrease of a predominant white blood cell subset; polymorphonuclear neutrophils. In individuals exhibiting normal blood cell counts, neutrophils constitute approximately 60% of the total leukocytes. However, approximately one in three cancer patients receiving chemotherapy suffer from neutropenia.

Hematopoietic growth factors are available on the market as recombinant proteins. These proteins include granulocyte colony stimulating factor (G-CSF) and granulocyte-macrophage colony stimulating factor (GM-CSF) for the treatment of neutropenia and erythropoietin (EPO) for the treatment of anemia. However, these recombinant proteins are expensive and subsequently their use is restricted and not readily available to all patients in need. Such post-therapeutic ameliorative treatments are unnecessary if patients are “chemoprotected” from immune suppression. International applications PCT/CA02/00535 and PCT/GB04/00457 describe the use of medium-chain length fatty acids, glycerides and analogues as chemoprotective agents also useful for the stimulation of hematopoiesis and treatment of neutropenia and anemia. However, none of the above growth factors or compounds can “chemoprotect” the patient and, at the same time, stimulate the patient's immune cell subset which most efficiently displays antitumor activity: cytotoxic T-lymphocytes (CTLs). Therefore, there is a need for novel compositions and methods to reduce the undesirable side effects of myelosuppressive states induced by chemotherapy and/or radiotherapy and, at the same time, maximally stimulate the immune system's antitumor response by stimulation of CTLs.

### SUMMARY OF THE INVENTION

A series of 6-substituted purinyl alkoxycarbonyl amino acids has been described in the literature for their ability to stimulate CTLs; B. Zacharie et al., Journal of Medicinal Chemistry, 40, 2883-2894 (1997) and S. Kadhim et al., International Journal of Immunopharmacology, 22, 659-671 (2000). A few of these compounds, in particular [[5-[6-(N,N-dimethylamino)purin-9-yl]pentoxy]-carbonyl]d-arginine, hereafter referred to as compound I, displayed an in vitro stimulation of CTLs comparable to the immune growth factor or cytokine interleukin 2. Furthermore, this potent stimulation of CTLs was shown to translate into significant in vivo antitumor activity. Two general points emerge from these articles:

(1) T-cells, in particular CTLs, are an important immune cell subset for mounting an antitumor response.

(2) A number of compounds can stimulate a specific immune response acting primarily on the T-cell lineage (T helper cells, CTLs) and they have antitumor activity. Such “thymomimetic” compounds include levamisole, methyl inosine monophosphate, isoprinosine, thymopentin and tucaresol.

However, neither of the articles, or the references cited therein, teach that prior art compounds which function as T-cell stimulants or thymomimetics (able to mimic the thymus) would, at the same time, be able to function as chemoprotective agents. In fact, the prior art teaches that these are specific compounds, especially compound I (hence the improvement) and so this is offered as an explanation for the relative lack of toxicity of this group of compounds compared to non-specific and subsequently toxic immunostimulants which directly stimulate multiple immune cell subsets. Examples of the latter include lipopolysaccharide and muramyl dipeptide.

It has been surprisingly discovered that 6-substituted purinyl compounds such as compound I possess chemoprotective activity as reflected by the ability to stimulate the proliferation of red blood cells and white blood cells. It has been further discovered that enhanced chemoprotective activity can be attained when the compounds of the present invention are combined with medium-chain fatty acids or metallic salts or triglycerides thereof or mono- or diglycerides.

### BRIEF DESCRIPTION OF THE DRAWINGS

FIG. 1 shows the effect of sodium caprate, compound I, and compound I in combination with sodium caprate on peripheral blood red cell count.

FIG. 2 shows the effect of sodium caprate, compound I, and compound I in combination with sodium caprate on bone marrow red cell count.

FIG. 3 shows the effect of sodium caprate, compound I, and compound I in combination with sodium caprate on bone marrow white cell count.

FIG. 4 shows the effect of sodium caprate, compound II, and compound II in combination with sodium caprate on peripheral blood red cell count.

FIG. 5 shows the effect of histamine dihydrochloride, compound I, and compound I in combination with histamine dihydrochloride on bone marrow white cell count.

FIG. 6 shows the effect of histamine dihydrochloride, compound I, and compound I in combination with histamine dihydrochloride on bone marrow red cell count.

FIG. 7 shows the effect of histamine dihydrochloride, compound II, and compound II in combination with histamine dihydrochloride on spleen white cell count.

### DETAILED DESCRIPTION OF CERTAIN EMBODIMENTS OF THE INVENTION

The present invention relates to a method of maintaining the patient's hematopoietic system while the patient is undergoing chemotherapy with cytotoxic drugs and/or radiotherapy with ionizing radiation for the treatment of cancer and, at the same time, stimulating the patient's CTLs so as to mount a more effective antitumor response.

The invention includes compounds, or pharmaceutically acceptable derivatives thereof, of the following general formula: 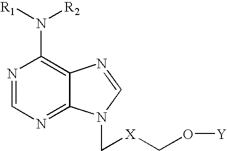
wherein
R₁═H, CH₃
R₂═H, CH₃, NH₂
R₁ may, or may not, equal R₂

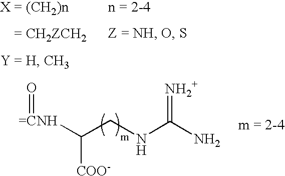

In one aspect of the present invention,

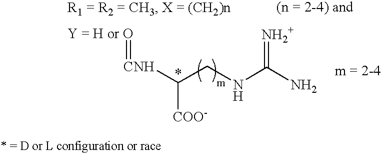

In a preferred aspect of this invention, 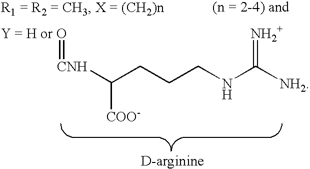
Particularly preferred are compounds I and II which have the following structures:

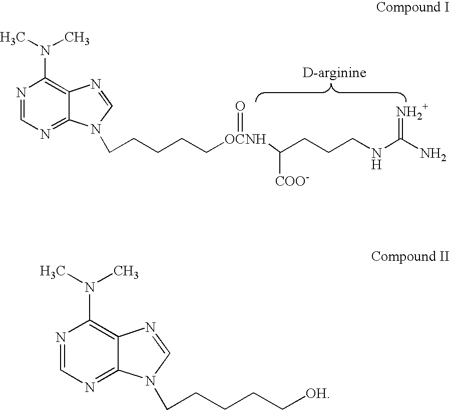

In another embodiment of this invention, compounds of the above formula may be used in combination with medium-chain fatty acids such as capric acid or caprylic acid or metallic salts or triglycerides thereof or mono- or diglycerides. Particularly preferred is the use the sodium or potassium salts of capric acid or caprylic acid or the triglyceride of capric acid (tricaprin) or caprylic acid (tricaprylin).

Medium-chain fatty acids refer to those fatty carboxylic acids with carbon chain lengths of 6 (hexanoic acid) to 12 (dodecanoic acid). Although saturated medium-chain fatty acids constitutes a preferred embodiment of this invention, this does not preclude the use of unsaturated medium-chain fatty acids. For example, 9-decenoic acid represents an example of an appropriate unsaturated medium-chain fatty acid which may be utilized in this invention. Amongst saturated fatty acids, caprylic acid (octanoic acid) and capric acid (decanoic acid) represent preferred medium-chain length fatty acids. Medium-chain triglycerides refer to the product which is obtained when three molecules of medium-chain fatty acids are esterified with one molecule of glycerol.

As described in the patent applications cited above, medium-chain fatty acids and their triglycerides are non-toxic materials which are used in the food and pharmaceutical industries. For example, in The Merck Index, 11^{th} Edition, 266 (1989) caprylic acid is reported to have an LD₅₀ (oral, rats)=10.08 g/kg which is essentially non-toxic. In fact, according to part 184 of the Code of Federal Regulations (CFR), the U.S. Food and Drug Administration has granted caprylic acid a GRAS (Generally Recognized As Safe) affirmation. Similarly, according to part 172 (CFR) free fatty acids (e.g., caprylic, capric) and their metallic salts are recognized as safe additives for use in food.

Although the combination of medium-chain fatty acids or metallic salts or triglycerides thereof with 6-substituted purinyl compounds represents a preferred embodiment of this invention, this does not preclude the use of the combination of other compounds, either described in prior art or not previously disclosed, which can stimulate hematopoiesis with 6-substituted purinyl compounds. For example, the nonapeptide SKF 107647 is reported to elicit an increase in neutrophils and monocytes, as described by C. Lyman et al., Antimicrobial Agents and Chemotherapy, 43, 2165-2169 (1999). Therefore, it is within the scope of this invention to use SKF 107647, or related peptides, with 6-substituted purinyl compounds. Similarly, it was observed that histamine can weakly stimulate hematopoiesis. However, the combination of a low dose of compound I with histamine results in a significant increase in white and red cell counts.

Compounds of the present invention, described by the above general formula, used alone or in combination with medium-chain fatty acids or their metallic salts or triglycerides thereof are able to stimulate the activity and increase the number of CTLs in the mammal. Although the literature cited above describes the ability to stimulate murine CTLs, subsequent experiments with human blood have revealed that compounds of the present invention can also stimulate human CTLs. Furthermore, this highly specific stimulation of CTLs achieved with these compounds is relatively non-toxic to the treated mammal. This is in contrast to other molecules which stimulate CTLs such as the cytokine interleukin 2. Interleukin 2 is a 15 kD endogeneous growth factor protein which efficiently stimulates CTLs. As such, it has been approved by the U.S. Food and Drug Administration for the treatment of renal cell carcinoma and melanoma. However, interleukin 2 has not been widely used because of severe toxicity which can accompany its use. Furthermore, as noted above, other molecules which non-specifically stimulate numerous immune cell subsets (macrophages, B- and NK cells as well as T-cells) also display toxicity, especially with prolonged use. However, compounds which specifically stimulate certain immune cell subsets tend to be relatively non-toxic. Thymomimetic compounds described above, for example, which mimic the thymus in their ability to specifically stimulate T-cells (T helper cells and CTLs) are relatively non-toxic. In prior art citations, it was therefore reasoned that the ability of 6-substituted purinyl compounds to selectively and specifically stimulate CTLs accounts for their unusual lack of toxicity. For example, the LD₅₀ (i.v., rat) for the thymomimetic compound levamisole is 16 mg/kg but for compound I it is more than 500 mg/kg.

It has been surprisingly discovered that the lack of toxicity of 6-substituted purinyl compounds is not completely accounted for by the ability to specifically stimulate CTLs. In fact, these compounds possess chemoprotective activity as reflected by their ability to stimulate the production of red blood cells and white blood cells. This is illustrated in Example I where a significant increase is observed in spleen red and white cell count in animals treated with the cytotoxic drug cyclophosphamide but pre-treated with compound I. In the same example, a significant increase is also observed in peripheral white blood cell count in the same animals treated with compound I. However, as shown in Example 2, pre-treatment with a combination of compound I and sodium caprate results in a significant increase in peripheral red blood cells. The chemoprotective activity of compound I has also been illustrated by the improved survival of animals in experiments wherein mice with MC-38 colon cancer were treated with compound I plus a toxic regimen of the cytotoxic drug 5-fluorouracil compared to mice treated with specific doses (e.g., 40 mg/kg) of 5-fluorouracil alone.

The 6-substituted purinyl compounds of the present invention may be prepared by the use of synthetic methods well known in the art. Thus, for example, it is possible to follow the synthetic procedure described in the above citation, Journal of Medicinal Chemistry, 40, 2883-2894, 1997. A similar detailed procedure is also provided in U.S. Pat. No. 5,994,361 issued Nov. 30, 1999. Similarly, the metallic salts of medium-chain fatty acids may be prepared by the synthetic procedure described in international application PCT/GB04/03182. Medium-chain fatty acids and triglycerides thereof, suitable for human use, are commercially available products which may be obtained from any one of a number of suppliers.

When used in cancer chemotherapy and/or radiotherapy, 6-substituted purinyl compounds, either alone or in combination with medium-chain fatty acids or metallic salts or triglycerides thereof, can be administered before, during and/or after chemotherapy and/or radiotherapy (e.g., within 14 days). By use of such compounds which stimulate an antitumor immune response and also which stimulate proliferation and subsequent restoration of hematopoietic cells, it is an intention of this invention to shorten the toxicity (leukopenia, neutropenia, anemia) associated with chemotherapy and/or radiotherapy. Such a reduction in toxicity opens the possibility of increasing the dose of cytotoxic drug and/or ionizing radiation, if such is deemed necessary by the clinician. With regard to the use of a combination of 6-substituted purinyl compounds with medium-chain fatty acids or metallic salts or triglycerides thereof, it is possible to mix and administer both components of the combination either separately or together. In the former case, the separate components of the combination may be administered at the same or different times in the treatment cycle and at the same or different times relative to chemotherapy and/or radiotherapy. The separate components of the combination may be administered by the same or different routes. Regardless, if given separately or together, components of the combination may be given by oral, sublingual, inhalation (nose spray), intravenous, intramuscular or subcutaneous routes.

It will be appreciated by those skilled in the art that the 6-substituted purinyl compounds of the present invention, either alone or in combination with medium-chain fatty acids or metallic salts or triglycerides thereof, include all pharmaceutically acceptable derivatives and analogues (including prodrugs) thereof, as well as all isomers and enantiomers. Furthermore, these compounds, either alone or in combination with medium-chain fatty acids or metallic salts or triglycerides thereof, are used for the manufacture of a medicament.

Another aspect of the invention is the method of treatment of a mammal, preferably a human, comprising the step of administering a 6-substituted purinyl compound of the above general formula, either alone or in combination with medium-chain fatty acids or metallic salts or triglycerides thereof, or a pharmaceutical composition thereof for the treatment of immune deficiency and/or leukopenia and/or neutropenia and/or anemia and/or tumor growth. It will be appreciated by those skilled in the art that treatment extends to prophylaxis, including prevention of metastasis from a primary tumor, as well as treatment of an established tumor, in conjunction with chemotherapy and/or radiotherapy, or symptoms of a cancer. It will be further appreciated that the amount of compound to be used in treatment will vary not only with the particular compound selected but also with the route of administration, the nature and severity of the cancer being treated and the age and condition of the patient and will be ultimately at the discretion of the attendant clinician. In general, however, a suitable dose will be in the range from about 0.1 to about 200 mg/kg of body weight per day given alone or in combination with about 1.0 to about 500 mg/kg medium-chain fatty acid or metallic salt or triglyceride thereof. Preferably, doses will range from about 1.0 mg/kg to about 100 mg/kg of compound per day. More preferably, between about 10.0 mg/kg to about 50 mg/kg of compound per day.

### EXAMPLES

The following further illustrate the practice of this invention but are not intended to be limiting thereof.

### Example 1

### Chemoprotection studies: In Vivo Protection of Hematopoietic Cells by Compound I

Female C57BL/6 mice, 6 to 8 weeks old, were immunosuppressed by treatment with 200 mg/kg of cyclophosphamide (CY) administered intravenously at day 0. To examine the chemoprotective effect of compound I, mice were pre-treated intraperitoneally at day −3, −2 and −1 with 50 mg/kg of the compound. Mice were sacrificed at day +5 by cardiac puncture and cervical dislocation. Then, cell suspensions were prepared from thymus, spleen and bone marrow as follows.

Tissues were crushed in PBS buffer and contaminating erythrocytes were lysed in ACK buffer (155 mM NH₄Cl, 12 mM NaHCO₃, 0.1 mM EDTA, pH 7.3) for five minutes. Cells were then collected by centrifugation and washed three times in PBS and resuspended in tissue culture medium. Cells were counted with a Coulter counter.

A significant increase in spleen red and white cell counts was observed after pre-treatment with compound I in CY-treated mice (Table 1). Further, some treated animals returned to a “baseline level” in terms of the spleen red cell count as compared to non-immunosuppressed animals (control). Additionally, a significant increase in peripheral white blood cell count was observed in the presence of compound I.

TABLE 1
Effect of CY and CY + compound I on spleen red cell,
spleen white cell, and peripheral blood white cell count.

| | Spleen | Spleen | Peripheral blood |
|---|---|---|---|
| Cell count (10⁶) | red cells | white cells | white cells |
| Control | 497 ± 219 | 118 ± 17 | 5.4 ± 1.3 |
| CY | 341 ± 107 | 34. ± 9 | 1.3 ± 0.6 |
| CY + compound I | 468 ± 104 | 48 ± 5 | 2.6 ± 0.6 |
| P-value (relative to CY) | 0.03 | 0.009 | 0.004 |

### Example 2

### Chemoprotection studies: In Vivo Induction of Immune Cell Proliferation or Protection by the Combination of Sodium Caprate and Compound I

The effect of sodium caprate, compound I, and the combination of both compounds on in vivo induction of hematopoietic cell proliferation or protection was determined following the protocol described in Example 1. Oral administration of sodium caprate (60.5 mM) and/or peritoneal injection of compound I (50 mg/kg) were performed on day −3, −2 and −1. Treated animals were compared to their respective control groups: CY+sodium caprate was compared to CY-PO (CY+PBS per os); CY+compound I was compared to CY-IP (CY+PBS intraperitoneal injection); and CY+sodium caprate+compound I was compared to CY-POIP (CY+PBS per os and PBS by intraperitoneal injection).

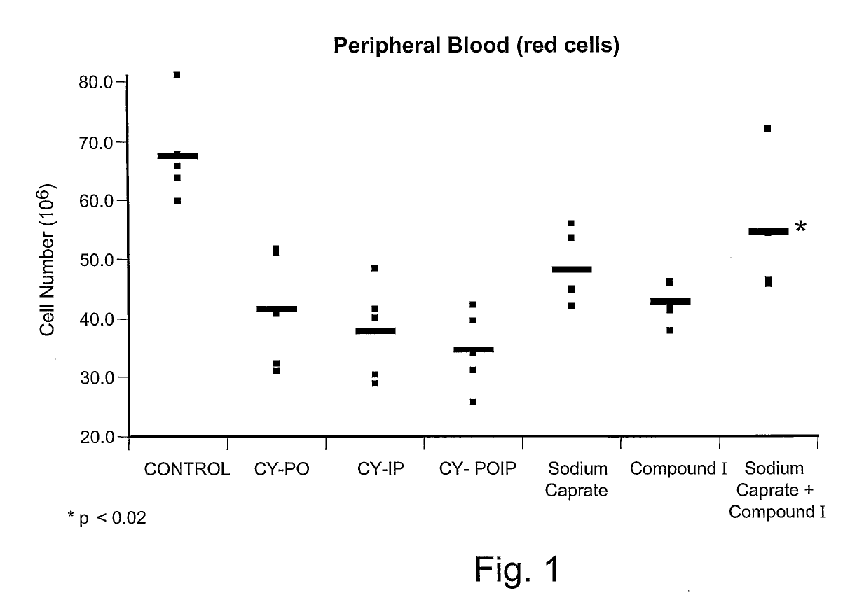

FIG. 1 represents the effect of sodium caprate, compound I, and the combination of both compounds on peripheral red blood cell count. A significant increase of peripheral red blood cells was obtained with pre-treatment with sodium caprate in CY-treated mice (compared to CY-per os control). A significant increase of peripheral red blood cells was also observed when CY-immunosuppressed mice were treated with compound I (compared to CY-i.p. control). Further, a combination therapy with sodium caprate and compound I resulted in an additive effect on the increase of peripheral red blood cell count. Additionally, some treated animals in the combination treatment return to a “baseline level” in terms of the peripheral red blood cell count as compared to non-immunosuppressed animals (control). Similar results were obtained when this experiment was repeated with the same dose of compoundI and a lower dose (6.05 mM) of sodium caprate.

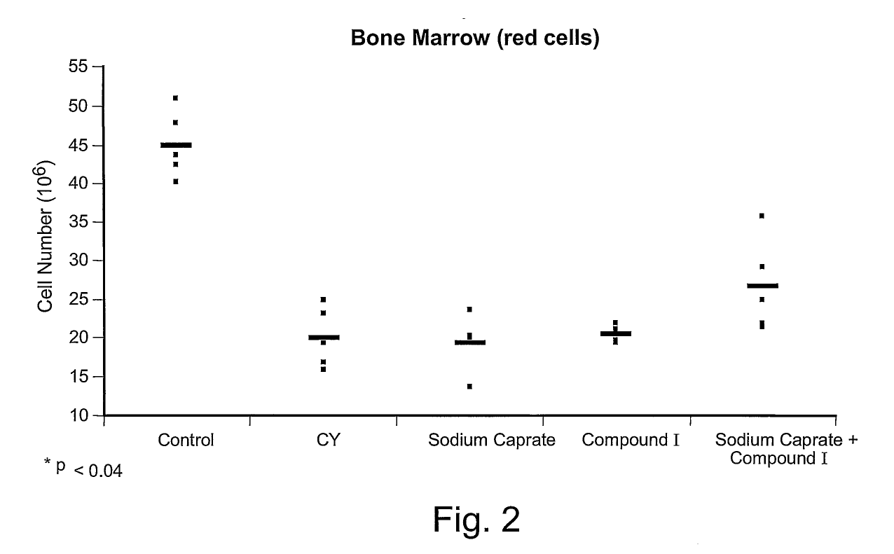

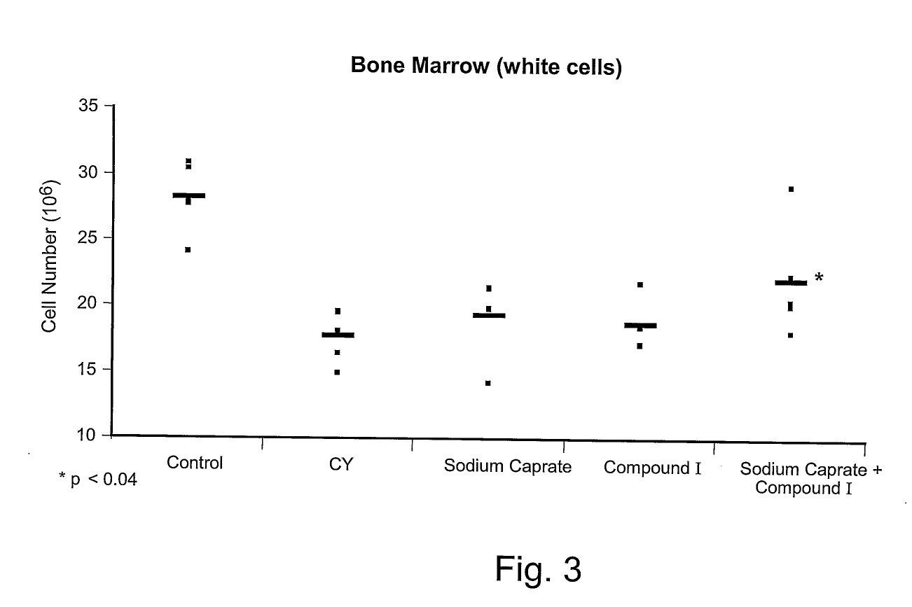

Effect of low dose of sodium caprate, compound I, and the combination of both compound was undertaken following the protocol described above with the exception that the compounds were intravenously administered at 1.21 mM for sodium caprate and 5 mg/kg for compound I. FIGS. 2 and 3 represents the effect of sodium caprate, compound I, and the combination of both compounds on bone marrow red and white cell count. No effect was observed when sodium caprate or compound I was used alone. However, a significant increase of bone marrow red (p<0.04) and white (p<0.04) cells was obtained when sodium caprate and compound I were used together. Further, combination therapy with sodium caprate and compound I resulted in a synergistic effect on the increase of bone marrow red and white cell count.

### Example 3

### Chemoprotection Studies: In Vivo Induction of Immune Cell Proliferation or Protection by the Combination of Tricaprin and Compound I

The effect of tricaprin, compound I, and the combination of both compounds on in vivo induction of hematopoietic cell proliferation or protection was determined following the protocol described in Example 1. Oral administration of tricaprin (60.5 mM) and/or peritoneal injection of compound I were performed on day −3, −2 and −1.

Table 2 represents the effect of tricaprin, compound I, and the combination of both compounds on bone marrow red cell count. A significant increase of bone marrow red cells was obtained by pre-treatment with a combination of tricaprin and compound I in CY-treated mice. This was a synergistic effect as compared to CY alone. Furthermore, mice treated with the combination of tricaprin and compound I demonstrated an increase (3 times) in CFU-GEMM cell population in bone marrow (Table 3).

TABLE 2
Effect of tricaprin, compound I, and compound I in combination
with tricaprin on bone marrow red cell count.

| Treatment | Cell count (10⁶) | P/control | P/CY |
|---|---|---|---|
| Control | 52.4 ± 10.3 | | |
| CY | 31.5 ± 3.5 | 0.004 | |
| CY + Tricaprin (60.5 mM) | 33.8 ± 3.7 | | 0.171 |
| CY + Compound I (50 mg/kg) | 37.4 ± 7.1 | | 0.075 |
| CY + Tricaprin + Compound I | 37.8 ± 2.8 | | 0.007 |

TABLE 3
Effect of tricaprin, compound I, and compound I in combination
with tricaprin on bone marrow progenitor cell.

| Treatment | CFU-GEMM (colony number) |
|---|---|
| Control | 0.5 |
| CY | 1 |
| CY + Tricaprin (60.5 mM) | 0.5 |
| CY + Compound I (50 mg/kg) | 1 |
| CY + Tricaprin + Compound I | 3 |

### Example 4

### Chemoprotection Studies: In Vivo Protection of Hematopoietic Cells by Compound II

The effect of compound II on in vivo induction of hematopoietic cell proliferation or protection was determined following the protocol described in Example 1. Oral administration of compound II (50 mg/kg or 100 mg/kg) was performed on day −3, −2 and −1.

A significant increase in bone marrow red and white cell counts was observed with pre-treatment with compound II in CY-treated mice (Table 4). Further, some treated animals return to a “baseline level” in terms of the bone marrow red and white cell counts as compared to non-immunosuppressed animals (control).

TABLE 4
Effect of CY and CY + compound II on
bone marrow red and white cell count.

| | Bone marrow | Bone marrow |
|---|---|---|
| Cell count (10⁶) | red cells | white cells |
| Control | 43.4 ± 4.6 | 21.6 ± 2 |
| CY | 31.6 ± 5.0 | 19.8 ± 4.4 |
| CY + compound II (50 mg/kg) | 29.4 ± 2.7 | 17.4 ± 3.5 |
| CY + compound II (100 mg/kg) | 43.9 ± 16.3 | 30.5 ± 9.4 |
| P-value (relative to CY) | 0.08 | 0.03 |

### Example 5

### Chemoprotection Studies: In Vivo Induction of Immune Cell Proliferation or Protection by the Combination of Sodium Caprate and Compound II

The effect of sodium caprate, compound II, and the combination of both compounds on in vivo induction of hematopoietic cell proliferation or protection was determined following the protocol described in Example 1. Oral administration of sodium caprate (60.5 mM) and/or compound II (50 mg/kg or 100 mg/kg) was performed on day −3, −2 and −1.

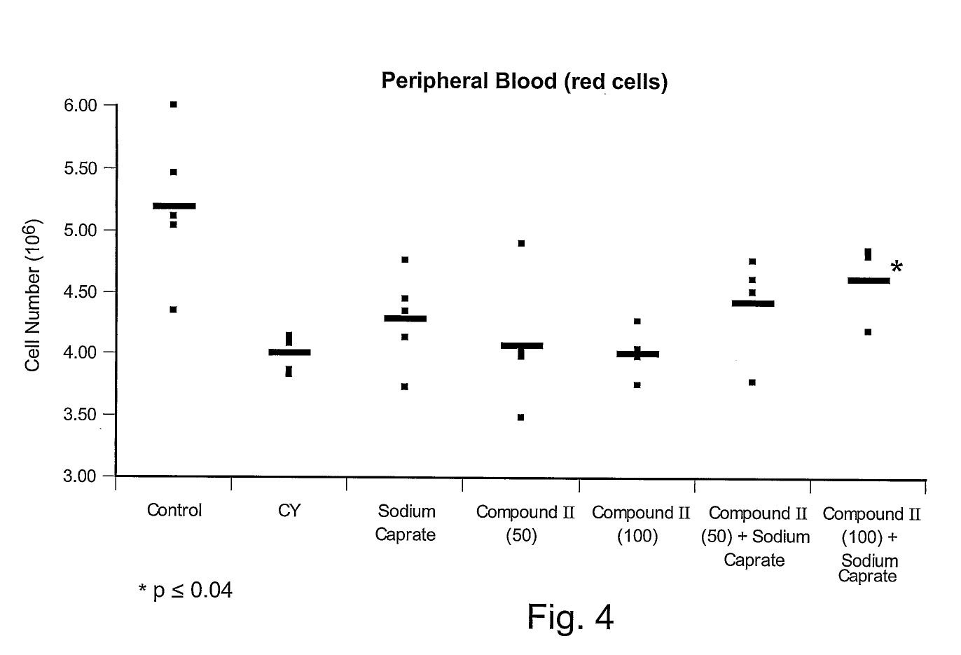

FIG. 4 represents the effect of sodium caprate, compound II, and the combination of both compounds on peripheral red blood cell count. A weak increase in peripheral red blood cells was obtained with pre-treatment with sodium caprate in CY-treated mice (compared to CY-per os control). However, combination therapy with sodium caprate and compound II resulted in a synergistic effect in the increase of peripheral red blood cells. Additionally, some treated animals in the combination treatment return to a “baseline level” in terms of the peripheral red blood cell count as compared to non-immunosuppressed animals (control).

### Example 6

### Chemoprotection Studies: In Vivo Induction of Immune Cell Proliferation or Protection by the Combination of Histamine Dihydrochloride and Compound I

Effect of low dose of histamine, compound I, and the combination of both compounds on in vivo induction of hematopoietic cell proliferation or protection was undertaken following the protocol described in Example 1. Intravenous administration of histamine dihydrochloride (25 mg/kg) and compound I (5 mg/kg) were performed on day −3, −2 and −1.

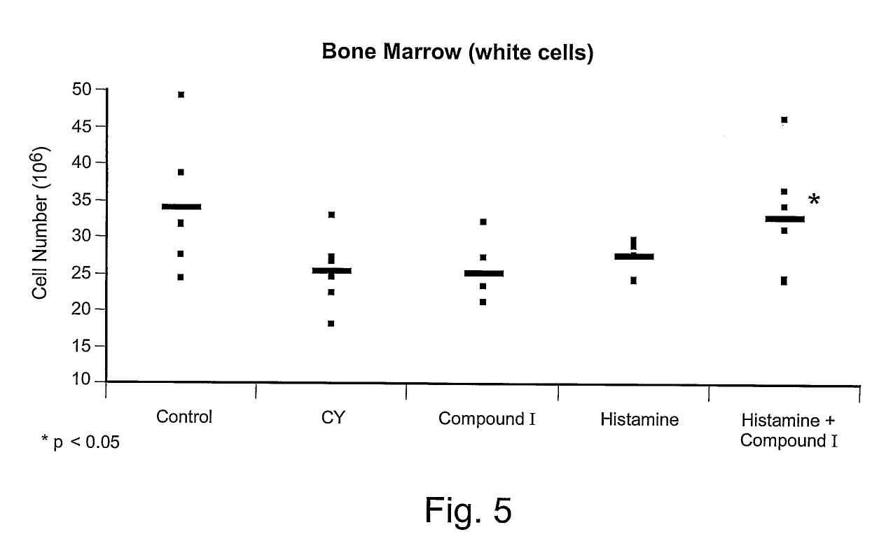

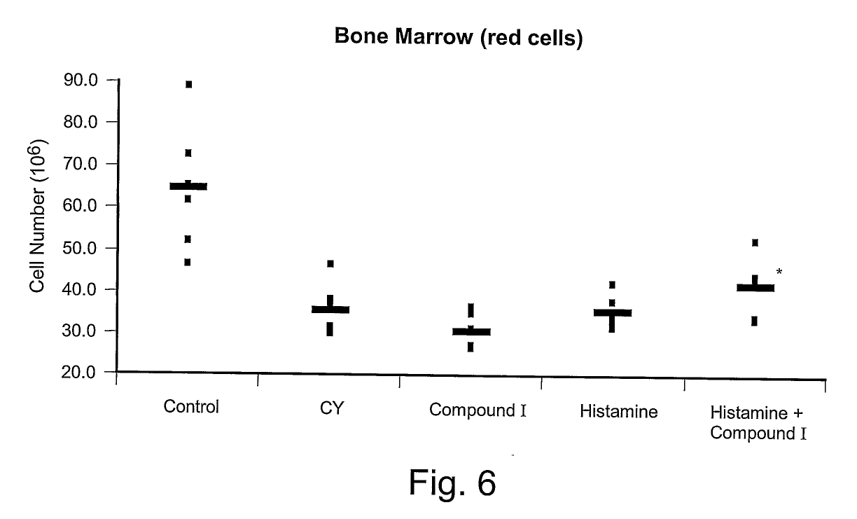

FIG. 5 represents the effect of histamine, compound I, and the combination of histamine and compound I on bone marrow white cell count. No significant effect was observed at low dose histamine and/or compound I in CY-treated mice (compared to CY). However, combination therapy with histamine and compound I resulted in a synergistic effect on the increase of bone marrow white cell count (FIG. 5). Additionally, some treated animals in the combination treatment return to a “baseline level” in terms of the bone marrow white cell count as compared to non-immunosuppressed animals (control). Further, a weak increase of bone marrow red cells was obtained with combination of histamine and compound I in CY-treated mice (FIG. 6).

### Example 7

### Chemoprotection Studies: In Vivo Induction of Immune Cell Proliferation or Protection by the Combination of Histamine Dihydrochloride and Compound II

Effect of low dose of histamine, compound II, and the combination of both compounds on in vivo induction of hematopoietic cell proliferation or protection was undertaken following the protocol described in Example 1. Intravenous administration of histamine dihydrochloride (25 mg/kg) and oral administration of compound II (100 mg/kg) were performed on day −3, −2 and −1.

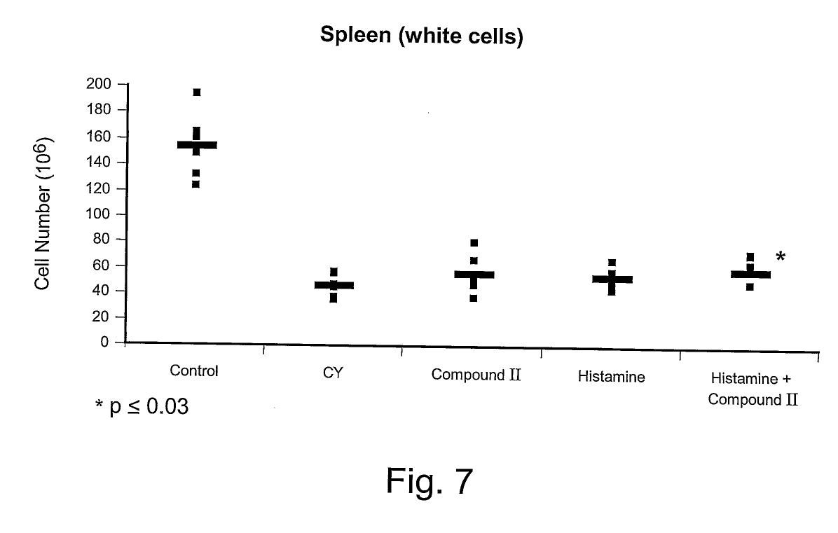

FIG. 7 represents the effect of histamine, compound II, and the combination of histamine and compound II on spleen white cell count. No significant effect was observed at low dose histamine and/or compound II in CY-treated mice (compared to CY). However, combination therapy with histamine and compound II resulted in a synergistic effect on the increase of spleen white cell count.

Patents, patent applications, and other publications cited herein are incorporated by reference in their entirety.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of the three transitions can be used to claim the invention.

It should be understood that an element described in this specification should not be construed as a limitation of the claimed invention unless it is explicitly recited in the claims. Thus, the claims are the basis for determining the scope of legal protection granted instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of the individual elements disclosed herein are considered to be aspects of the invention; similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1-16. (canceled)

17. A method of treating anemia and/or neutropenia in a cancer patient in need of treatment as a result of chemotherapy and/or radiotherapy, comprising administration of a pharmacologically effective amount of a composition comprising one or more compounds described by the following general formula: 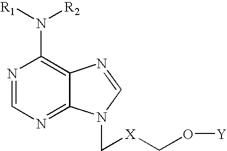
wherein
R₁═H, CH₃
R₂═H, CH₃, NH₂
R₁ may, or may not, equal R₂

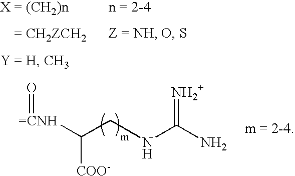

18. The method according to claim 17, wherein said composition comprises one or more compounds described by said general formula with Y═H or CH₃ and is orally administered.

19. The method according to claim 17, wherein said composition comprises one or more compounds described by said general formula with R₁═R₂═CH₃, X═(CH₂)n (n=2-4) and

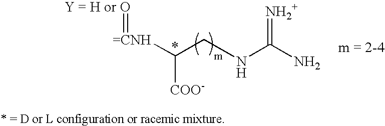

20. The method according to claim 17, wherein said composition comprises one or more compounds described by said general formula with R₁═R₂═CH₃, X═(CH₂)n (n=2-4) and Y═H or D-arginine.

21. The method according to claim 17, wherein said composition is further comprised of medium-chain length fatty acids H₃C(CH₂)nCOOH (n=4-10) or metallic salts or triglycerides thereof.

22. The method according to claim 17, wherein said composition is further comprised of sodium caprylate or sodium caprate or caprylic acid or capric acid or tricaprylin or tricaprin.

23. The method according to claim 17, wherein anemia arising from chemotherapy is at least prevented, inhibited, or reduced.

24. The method according to claim 17, wherein anemia arising from radiotherapy is at least prevented, inhibited, or reduced.

25. The method according to claim 17, wherein neutropenia arising from chemotherapy is at least prevented, inhibited, or reduced.

26. The method according to claim 17, wherein neutropenia arising from radiotherapy is at least prevented, inhibited, or reduced.

27. The method according to claim 17, wherein said composition is further comprised of histamine.

28. A method of making one or more compounds for treating anemia and/or neutropenia in a cancer patient in need of treatment as a result of chemotherapy and/or radiotherapy, wherein said compounds are described by the following general formula: 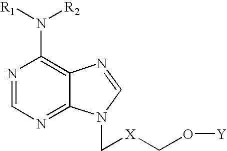
wherein
R₁═H, CH₃
R₂═H, CH₃, NH₂
R₁ may, or may not, equal R₂

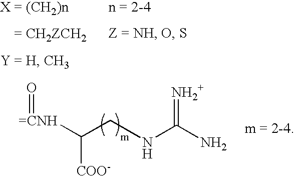

29. A composition for treating anemia and/or neutropenia in a cancer patient in need of treatment as a result of chemotherapy and/or radiotherapy, wherein said composition comprises a pharmacologically effective amount of one or more compounds described by the following general formula: 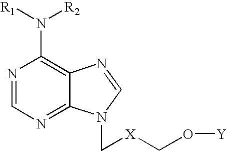
wherein
R₁═H, CH₃
R₂═H, CH₃, NH₂
R₁ may, or may not, equal R₂

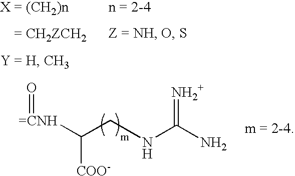

30. The composition of claim 29, wherein said composition comprises one or more compounds described by said general formula with Y═H or CH₃.

31. The composition of claim 29, wherein said composition comprises one or more compounds described by said general formula with R₁═R₂═CH₃, X═(CH₂)n (n=2-4) and

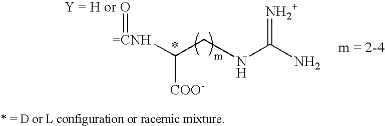

32. The composition of claim 29, wherein said composition comprises one or more compounds described by said general formula with R₁═R₂═CH₃, X═CH₂)n (n=2-4) and Y═H or D-arginine.

33. The composition of claim 29, wherein said composition is further comprised of medium-chain length fatty acids H₃C(CH₂)nCOOH (n=4-10) or metallic salts or triglycerides thereof.

34. The composition of claim 29, wherein said composition is further comprised of sodium caprylate or sodium caprate or caprylic acid or capric acid or tricaprylin or tricaprin.

35. The composition of claim 29, wherein said composition is further comprised of histamine.
